(19) United States
(12) Patent Application Publication
(10) Pub. No.: US 2024/0386989 A1
(43) Pub. Date: Nov. 21, 2024

(54) AMINO ACID SEQUENCE INFILLING

(71) Applicant: INTERNATIONAL BUSINESS MACHINES CORPORATION, Armonk, NY (US)

(72) Inventors: Payel Das, Yorktown Heights, NY (US); Devleena Das, Atlanta, GA (US); Pin-Yu Chen, White Plains, NY (US); Inkit Padhi, White Plains, NY (US); Amit Dhurandhar, Yorktown Heights, NY (US); Igor Melnyk, White Plains, NY (US); Enara C. Vijil, Westchester, NY (US)

(21) Appl. No.: 18/319,441

(22) Filed: May 17, 2023

(51) Int. Cl. G16B 5/20; G06N 20/00

(52) U.S. Cl. CPC G16B 5/20; G06N 20/00

(57) ABSTRACT

A first language vector can be generated by performing a first linear projection on a partial amino acid sequence vector. A second language vector can be generated by performing natural language processing on the first language vector. A predicted amino acid sequence vector can be generated by performing a second linear projection on the second language vector. A complete amino acid sequence listing can be output based on the predicted amino acid sequence vector.

### STATEMENT REGARDING PRIOR DISCLOSURES BY THE INVENTOR OR A JOINT INVENTOR

The following disclosure(s) are submitted under 35 U.S.C. 102(b)(1)(A):

DISCLOSURE(S): Melnyk et al., Reprogramming Large Pretrained Language Models for Antibody Sequence Infilling, CoRR 2022, pp. 1-18 (Sep. 28, 2022).

### BACKGROUND

The present invention relates to data processing systems, and more specifically, to use of artificial intelligence and machine learning to effect treatment for diseases or medical conditions.

Proteins are large, complex molecules that perform many critical functions in the body. Proteins are made up of smaller units called amino acids. Amino acids are attached to one another in long chains. Antibodies are a type of protein essential for treatment of many diseases. Antibodies are produced by the immune system in response to the presence of foreign substances, called antigens, in the body. Antibodies circulate in the blood and bind to antigens, which helps to destroy antigens. Some antibodies even destroy antigens directly.

Antibody molecules roughly are Y-shaped molecules that include paired heavy and light polypeptide chains forming amino acid sequences. Amino acid sequences are studied for various medical purposes. For example, amino acid sequences are for use in designing new antibodies (e.g., monoclonal antibodies and polyclonal antibodies), which may be made in laboratories, for use in treating diseases. Such man-made antibodies can target specific antigens, which can improve treatment of particular diseases. Moreover, some man-made antibodies can be used to stimulate the body's immune response and its own production of antibodies.

### SUMMARY

A method can include generating, using a processor, a first language vector by performing a first linear projection on a partial amino acid sequence vector. The method also can include generating a second language vector by performing natural language processing on the first language vector. The method also can include generating a predicted amino acid sequence vector by performing a second linear projection on the second language vector. The method also can include outputting a complete amino acid sequence listing based on the predicted amino acid sequence vector.

A system includes a processor programmed to initiate executable operations. The executable operations can include generating a first language vector by performing a first linear projection on a partial amino acid sequence vector. The executable operations also can include generating a second language vector by performing natural language processing on the first language vector. The executable operations also can include generating a predicted amino acid sequence vector by performing a second linear projection on the second language vector. The executable operations also can include outputting a complete amino acid sequence listing based on the predicted amino acid sequence vector.

A computer program product includes a computer readable storage medium having program code stored thereon. The program code is executable by a data processing system to initiate operations. The operations can include generating a first language vector by performing a first linear projection on a partial amino acid sequence vector. The operations also can include generating a second language vector by performing natural language processing on the first language vector. The operations also can include generating a predicted amino acid sequence vector by performing a second linear projection on the second language vector. The operations also can include outputting a complete amino acid sequence listing based on the predicted amino acid sequence vector.

This Summary section is provided merely to introduce certain concepts and not to identify any key or essential features of the claimed subject matter. Other features of the inventive arrangements will be apparent from the accompanying drawings and from the following detailed description.

### BRIEF DESCRIPTION OF THE DRAWINGS

FIG. 1 is a block diagram illustrating an example of a network data processing system.

FIG. 2 is a block diagram illustrating example architecture for an amino acid sequence infilling model.

FIG. 3 depicts a block diagram illustrating machine training of an example AASIM 200 according to an embodiment of the present invention.

FIG. 4 is a flowchart illustrating an example of a method of amino acid sequence infilling.

### DETAILED DESCRIPTION

The present invention relates to data processing systems, and more specifically, to use of artificial intelligence and machine learning to effect treatment for diseases or medical conditions.

The arrangements described herein are directed to computer technology, and provide an improvement to computer technology. Specifically, the present arrangements utilize machine learning and artificial intelligence (AI) to improve efficiency and accuracy of a data processing system at determining missing elements of amino acid sequences.

Amino acid sequences of proteins, such as antibodies, can be studied by performing protein sequencing on the proteins to generate listings of the amino acid sequences. The goal of protein sequencing is to identify each amino acid, and the order of amino acids, in each amino acid chain of the protein. Protein sequencing is a complex process, though, and sometimes amino acids in portions of amino acid chains are not identified. Accordingly, an amino acid sequence of a protein may be incomplete and only partially known.

Notwithstanding, in accordance with the arrangements described herein, AI and machine learning can be implemented to train an amino acid sequence infilling model to determine the amino acids missing from the amino acid sequence listings, and determine their placements in the amino acid sequences. The amino acid sequence infilling model can be used to process partial amino acid sequence listings and determine their missing amino acids. The amino acid sequence infilling model can output predicted amino acid sequence listings that include the amino acids determined to be the missing, and listed in the correct order, for the amino acid chains. In this regard, the amino acid sequence infilling model can receive as input a partial amino acid sequence listing of an amino acid, and output a complete amino acid sequence listing of the amino acid.

A main challenge of antibody design is generating the complementary-determining regions (CDRs) for antibodies. CDRs are part of the antibody amino acid chains where the antibodies bind to specific antigens. The design of a CDR may be heavily influenced by, or even copied from, a CDR in a naturally occurring antibody. If some amino acids in the natural CDR are unknown, designing an effective antibody based on that CDR can be very challenging. Thus, generating a complete amino sequence listing for the amino acid containing the CDR can improve antibody design.

By way of example, treatment of a particular disease, for example a particular form of cancer, can be improved by designing antibodies that target specific antigens of the disease. In illustration, a monoclonal antibody can be designed to bind to a specific antigen of a cell of a particular form of cancer, thus providing targeted treatment for that cancer. That monoclonal antibody can serve as a flag to attract disease-fighting molecules or serve as a trigger that promotes cell destruction by other immune system processes. Thus, generating a complete amino sequence listing for an antibody to be used for designing a new antibody effects a particular treatment for a disease.

Experimental results achieved by implementing the arrangements described herein indicate that the present arrangements provide significant improvement over other technologies that have been used in attempts to determine the amino acids missing from the amino acid sequence listings. In this regard, the present arrangements provide higher accuracy in predicting CDRs.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as amino acid sequence infilling model (AASIM) 200 and an AASIM training module 300. In addition to blocks 200, 300, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and blocks 200, 300, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

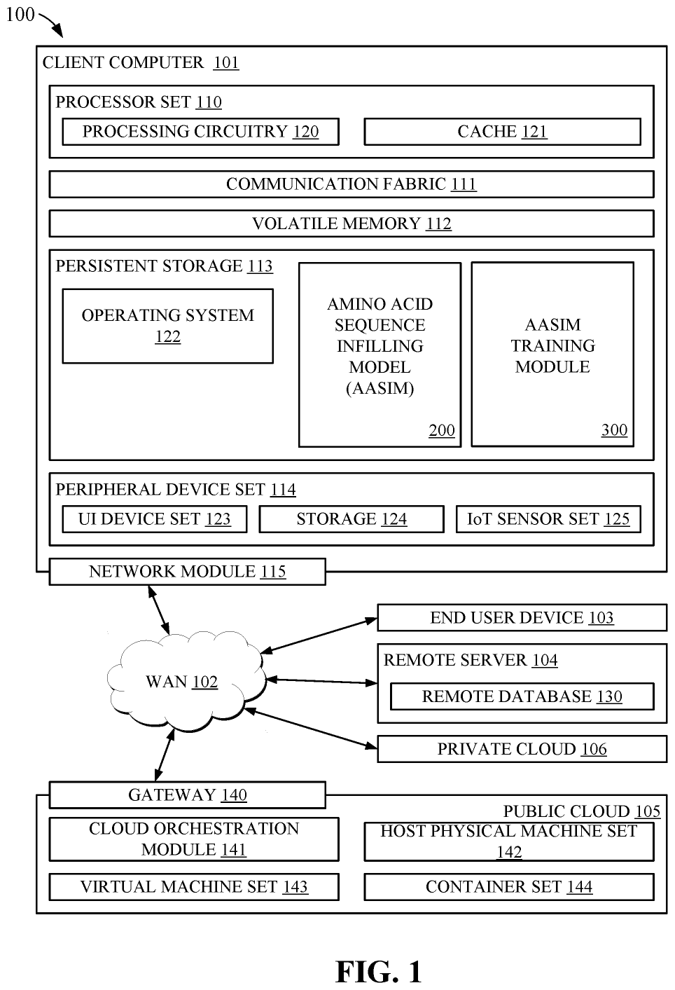

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in blocks 200, 300 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in blocks 200, 300 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

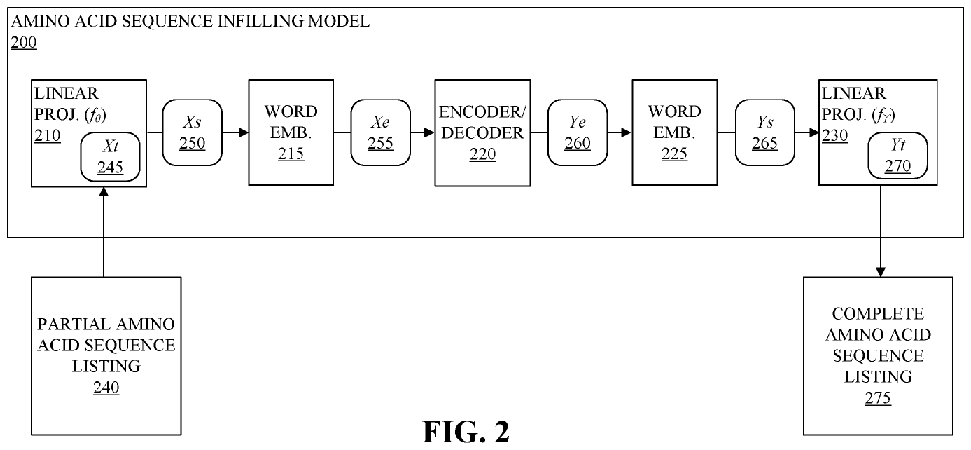

FIG. 2 is a block diagram illustrating example architecture for an amino acid sequence infilling model (AASIM) 200. AASIM 200 can include a first linear projector 210, a first word embedder 215, an encoder/decoder 220, a second word embedder 225 and a second linear projector 230. In one or more arrangements, first word embedder 215, encoder/decoder 220 and second word embedder 225 can be implemented using a Bidirectional Encoder Representations from Transformers (BERT) language model. BERT is a family of masked-language models used in Natural Language Processing (NLP). The BERT language model can include one or more ANNs trained using AI and machine learning to perform NLP.

In operation, AASIM 200 can receive a partial amino acid sequence listing 240. The following Table 1 is listing is an example of a partial amino acid sequence listing 240:

TABLE 1
VQLVESGGGLVQPGGSLRLSCAAS********MSWVRQAPG
KGLEWVSA*******YYADSVKGRFTISRHNSKNTLYLQMK
SLRPEDTAIYYC*******************WGQGTMVTVSSAST
KGPSVFPLAPGGTAALGCLVKDYFPEPVTVSWNSGALTSG
VHTFPAVLQSSGLYSLSSVVTVPSSSLGTQTYICNVNHKPS
NTKVDKKVEP

In this example, the asterisks (*) represent amino acids missing from the partial amino acid sequence listing 240.

First linear projector 210 can represent the partial amino acid sequence listing 240 as a partial amino acid sequence vector (Xₜ) 245, where:

$${X}_{t} = 〈 a 1 , a 2 , … , {a}_{n} 〉$$

aᵢ: tokens representing amino acids in protein vocabulary
   First linear projector 210 can replace any missing amino acids (*) with a particular token designated to represent missing amino acids.

First linear projector 210 can perform a linear projection (f_{θ}) on partial amino acid sequence vector (Xₜ) 245 to generate a first language vector (Xₛ) 250. First language vector (Xₛ) 250 can be a mapping of the tokens (a) in partial amino acid sequence vector (Xₜ) 245 to natural language words, where:

$${X}_{s} = 〈 {w}_{1} , {w}_{2} , … , {w}_{n} 〉$$

wᵢ: word tokens
   θ can be a linear projection matrix. E.g.,:
f_{θ}: Xₜ→Xₛ
where:
Xₛ=Xₜθ
θ∈ [symbol] ^{|Vt|×|Vs|}
Vₜ: protein vocabulary tokens
Vₛ: original vocabulary tokens

The size of the linear projection matrix θ can be Vₜ×Vₛ. The total number of possible tokens, Vₜ, in the protein vocabulary can be thirty. For example, twenty tokens can represent respective amino acids, and ten tokens can represent respective auxiliary variables. The total number of possible tokens, Vₛ, in the original vocabulary can be based on the natural language used for the original vocabulary. For example, if the original vocabulary is English, the total number of possible tokens, Vₛ, in the original vocabulary can be 30,522. Thus, in the present example, the size of the linear projection matrix θ can be 30×30,522. It should be noted that the present arrangements are not limited to English as being the natural language used for the original vocabulary. In one or more arrangements the natural language used for the original vocabulary can be Spanish, French, Italian, German, Mandarin, etc.

First word embedder 215 can embed the words in first language vector (Xₛ) 250 into a first word embedding vector (Xₑ) 255. First word embedding vector (Xₑ) 255 can include a representation of each word contained in first language vector (Xₛ) 250. Each representation can be, for example, a real-valued vector that encodes the meaning of the corresponding word in such a way that words that are closer in the vector space are expected to be similar in meaning. In illustration, first word embedder 215 can use language modeling and feature learning techniques, where words or phrases from the original vocabulary are mapped to vectors of real numbers. A desired word or symbol (e.g., “UNK”) can be used to embed words, in first language vector (Xₛ) 250, that represent missing amino acids (*). The size of first word embedding vector (Xₛ) 255 can be Vₛ×h, where h is a hidden or latent dimension/space. The value of h can be 1,024, but the present arrangements are not limited in this regard.

First word embedder 215 can utilize, for example, WordPiece™ to embed the words in first language vector (Xₛ) 250 into first word embedding vector (Xₑ) 255. WordPiece™ is a subword segmentation algorithm used in NLP. Using WordPiece™, the vocabulary can be initialized with individual characters in the original language, then the most frequent combinations of symbols in the vocabulary can be iteratively added to the vocabulary. The process can include: initializing a word unit inventory with all the characters in the text; building a language model on the training data using the word unit inventory; generating a new word unit by combining two units out of the current word inventory to increment the word unit inventory by one; choosing the new word unit out of all the possible ones that increases the likelihood on the training data the most when added to the model. The building and generating steps can be repeated until a predefined limit of word units is reached or the likelihood increase falls below a certain threshold.

Encoder/decoder 220 can translate first word embedding vector (Xₑ) 255 into second word embedding vector (Yₑ) 260. In illustration, encoder/decoder 220 can include an encoder that reads text represented by first word embedding vector (Xₑ) 255 and a decoder that produces, from that text, sentence predictions for NLP, for example in accordance with the BERT language model. The sentence predictions can include predictions of words missing in first language vector (Xₛ) 250 (e.g., indicated as “UNK”), and represented accordingly in first word embedding vector (Xₑ) 255. From the sentence predictions, encoder/decoder 220 can generate second word embedding vector (Yₑ) 260. Second word embedding vector (Yₑ) 260 can include representations of words in the sentence predictions. As previously discussed, each representation can be, for example, a real-valued vector that encodes the meaning of the corresponding word in such a way that words that are closer in the vector space are expected to be similar in meaning. The size of second word embedding vector (Yₑ) 260 can be Vₛ×h.

Second word embedder 225 can translate second word embedding vector (Ye) 260 to second language vector (Ys) 265, where:

$${Y}_{s} = 〈 {w}_{1} , {w}_{2} , … , {w}_{n} 〉$$

wᵢ: word tokens
   In illustration, second word embedder 225 can use language modeling and feature learning techniques to map vectors of real numbers in second word embedding vector (Ye) 260 to words from the original vocabulary. Because second word embedding vector (Ye) 260 can be based on sentence predictions, second word embedding vector (Ye) 260 can include predicted words in place of words missing in first language vector (Xₛ) 250 (e.g., indicated as “UNK”). Second word embedder 215 can utilize, for example, WordPiece™ to translate second word embedding vector (Ye) 260 to second language vector (Ys) 265.

Collectively, the processes performed by first word embedder 215, encoder/decoder 220 and second word embedder 225 to arrive at second language vector (Ys) 265 from first language vector (Xₛ) 250 are an example of performing NLP. Such processes can be implemented using a natural language model, for example a BERT language model.

Second linear projector 230 can perform a linear projection (f_{γ}) on second language vector (Ys) 265 to generate a predicted amino acid sequence vector (Yₜ) 270. In illustration, second linear projector 230 can generate predicted amino acid sequence vector (Yₜ) 270 by mapping natural language words contained second language vector (Yₛ) 265 to predicted amino acid sequence vector (Yₜ) 270, where:

$${Y}_{t} = 〈 {a}_{1} , {a}_{2} , … , {a}_{n} 〉$$

a: tokens representing amino acids in protein vocabulary
   γ can be a linear projection matrix. E.g.,:
f_{γ}: Yₛ→Yₜ
where:
Yₜ=Yₛγ
γ∈ [symbol] ^{|Vs|×|Vt|}
Vₜ: protein vocabulary tokens
Vₛ: original vocabulary tokens
   Thus, predicted amino acid sequence vector (Yₜ) 270 can be determined by the following equation:

$${y}_{t} = {f}_{ϒ} ( M ( {f}_{θ} ( {X}_{t} ) ) )$$

where:
f_{γ}: Yₛ→Yₜ
M: natural language processing performed by BERT language model
f_{θ}: Xₜ→Xₛ
Xₜ: partial amino acid sequence vector
   The size of the linear projection matrix γ can be Vₛ×Vₜ.

Second linear projector 230 can convert predicted amino acid sequence vector (Yₜ) 270 to complete amino acid sequence listing 275, and AASIM 200 can output complete amino acid sequence listing 275. The following Table 2 is listing is an example of a complete amino acid sequence listing 275:

TABLE 2
VQLVESGGGLVQPGGSLRLSCAASGVTVSSNYMSWVRQA
PGKGLEWVSAVYSGGSTYYADSVKGRFTISRHNSKNTLYL
QMKSLRPEDTAIYYCARLINHYYDSSGDGGAFDIWGQGTM
VTVSSASTKGPSVFPLAPGGTAALGCLVKDYFPEPVTVSW
NSGALTSGVHTFPAVLQSSGLYSLSSVVTVPSSSLGTQTYIC
NVNHKPSNTKVDKKVEP

In this example, complete amino acid sequence listing 275 includes amino acids predicted to be the amino acids missing from the partial amino acid sequence listing 240 of Table 1.

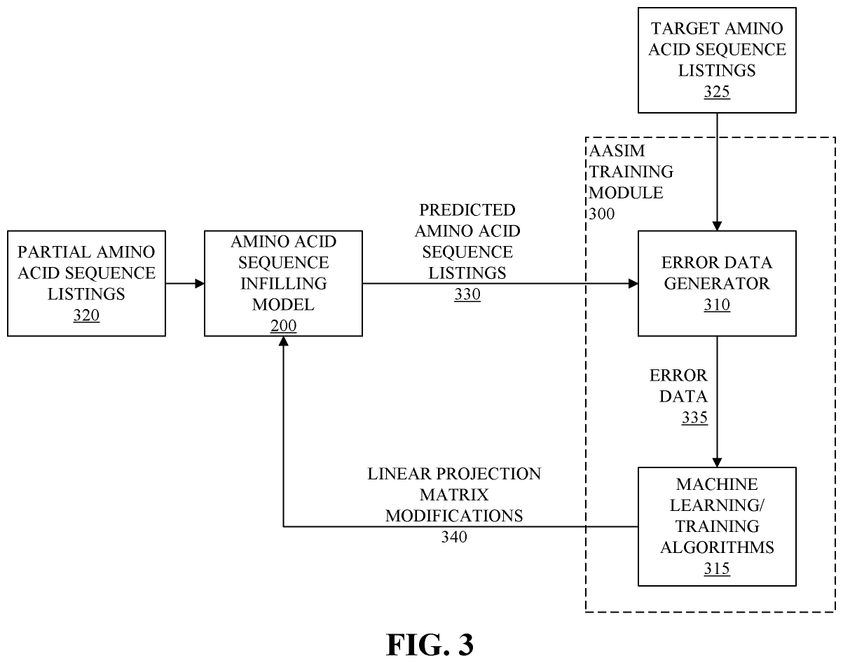

FIG. 3 depicts a block diagram illustrating machine training of an example AASIM 200 according to an embodiment of the present invention.

As noted, in one or more arrangements, first word embedder 215, encoder/decoder 220 and second word embedder 225 can be implemented using a BERT language model. The BERT language model can be pre-trained using AI and machine learning to perform NLP. However, additional training can be performed on AASIM 200 having the trained BERT language model integrated with AASIM 200. Specifically, AASIM 200 can be trained using AI and machine learning to predict amino acids missing from listings of amino acid sequences.

In illustration, the machine learning can train first linear projector 210 to perform linear projections (f_{θ}) on partial amino acid sequence vectors (Xₜ) 245 to generate first language vectors (Xₛ) 250. The machine learning also can train second linear projector 230 to perform linear projections (f_{γ}) on second language vectors (Ys) 265 to map natural language words contained second language vectors (Yₛ) 265 to predicted amino acid sequence vectors (Yₜ) 270. In this regard, the machine learning can train AASIM 200 by adjusting the linear projection matrices θ and γ.

AASIM training module 300 can include error data generator 310 and machine learning/training algorithms 315 configured to train AASIM 200 described herein. AASIM 200 can receive, as input training data for machine learning, partial amino acid sequence listings 320 for which the missing amino acids are known. For example, a partial amino acid sequence listing 320 can be an amino acid sequence in which one or more CDRs are masked.

For each partial amino acid sequence listing 320, there can be a target amino acid sequence listing 325 that includes each of the amino acids contained in the partial amino acid sequence listing 320 and each of the amino acids missing from the partial amino acid sequence listing 320. Moreover, the sequence of the amino acids in the target amino acid sequence listing 325 can be the same as the sequence of the amino acids in the partial amino acid sequence listing 320, with the addition of amino acids in place of the amino acids missing from the partial amino acid sequence listing 320. For example, target amino acid sequence listing 325 can include the same amino acid sequence as the partial amino acid sequence listing 320, but with the CDRs unmasked. In this regard, target amino acid sequence listing 325 can be used as a ground truth sequence (y*ₜ).

AASIM 200 can process a partial amino acid sequence listing 320, for example as previously described, and generate a predicted amino acid sequence listing 330 based on such processing. Error data generator 310 can compare predicted amino acid sequence listing 330 to target amino acid sequence listing 325, and generate error data 335 indicating differences between predicted amino acid sequence listing 330 and target amino acid sequence listing 325. Machine learning/training algorithms 315 can process the error data 335 and, based on such processing, generate linear projection matrix modifications 340. Linear projection matrix modifications 340 can modify coefficients of linear projection matrix θ of AASIM 200 and modify coefficients of linear projection matrix Y of AASIM 200, in a process known as machine learning (or training). For example, AASIM training module 300 can train linear projection matrix θ and linear projection matrix γ with respect to minimizing [symbol] _{N LL}(yₜ, y*ₜ), which is the loss between the predicted amino acid sequence vector, determined by yₜ=f_{γ}(M(f_{θ}(Xₜ))), and the partial amino acid sequence vector given the partial amino acid sequence listing 320 and the ground truth sequence (y*ₜ) (e.g., the target amino acid sequence listing 325).

The machine learning process can iterate until an objective is satisfied. Such objective can be, for example, generation of error data 335 having one or more values below one or more threshold values. In illustration, the objective can be generation of error data 335 having fewer than a threshold number of amino acid differences between predicted amino acid sequence listings 330 and target amino acid sequence listings 325. In one or more arrangements, the objective can be to iterate the machine learning process a threshold number of times, or the objective can be to iterate the machine learning process a threshold duration of time. Moreover, the machine learning process can be iterated at any desired time.

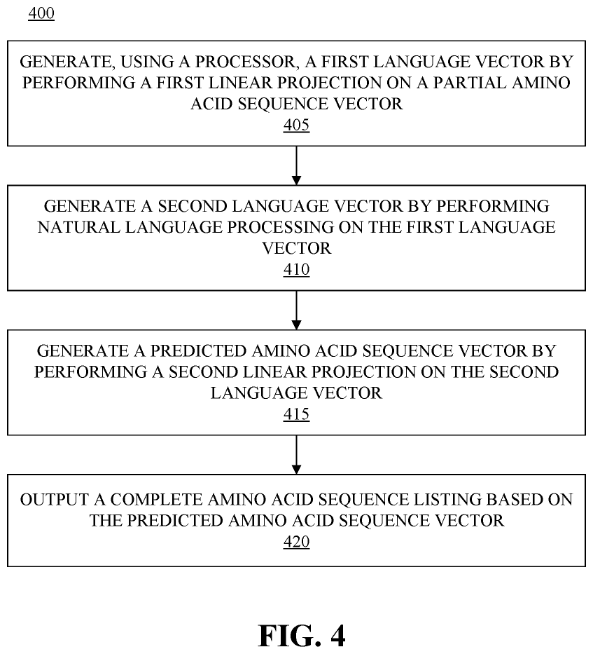

FIG. 4 is a flowchart illustrating an example of a method 400 of amino acid sequence infilling. Method 400 can be performed by AASIM 200.

At step 405 AASIM 200 can generate, using a processor, a first language vector by performing a first linear projection on a partial amino acid sequence vector.

At step 410 AASIM 200 can generate a second language vector by performing natural language processing on the first language vector.

At step 415 AASIM 200 can generate a predicted amino acid sequence vector by performing a second linear projection on the second language vector.

At step 420 AASIM 200 can output a complete amino acid sequence listing based on the predicted amino acid sequence vector.

The foregoing description is just an example of embodiments of the invention, and variations and substitutions. While the disclosure concludes with claims defining novel features, it is believed that the various features described herein will be better understood from a consideration of the description in conjunction with the drawings. The process(es), machine(s), manufacture(s) and any variations thereof described within this disclosure are provided for purposes of illustration. Any specific structural and functional details described are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “includes,” “including,” “comprises,” and/or “comprising,” when used in this disclosure, specify the presence of stated features, integers, steps, operations, elements, and/or components, but do not preclude the presence or addition of one or more other features, integers, steps, operations, elements, components, and/or groups thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “includes,” “including,” “comprises,” and/or “comprising,” when used in this disclosure, specify the presence of stated features, integers, steps, operations, elements, and/or components, but do not preclude the presence or addition of one or more other features, integers, steps, operations, elements, components, and/or groups thereof.

Reference throughout this disclosure to “one embodiment,” “an embodiment,” “one arrangement,” “an arrangement,” “one aspect,” “an aspect,” or similar language means that a particular feature, structure, or characteristic described in connection with the embodiment is included in at least one embodiment described within this disclosure. Thus, appearances of the phrases “one embodiment,” “an embodiment,” “one arrangement,” “an arrangement,” “one aspect,” “an aspect,” and similar language throughout this disclosure may, but do not necessarily, all refer to the same embodiment.

The term “plurality,” as used herein, is defined as two or more than two. The term “another,” as used herein, is defined as at least a second or more. The term “coupled,” as used herein, is defined as connected, whether directly without any intervening elements or indirectly with one or more intervening elements, unless otherwise indicated. Two elements also can be coupled mechanically, electrically, or communicatively linked through a communication channel, pathway, network, or system. The term “and/or” as used herein refers to and encompasses any and all possible combinations of one or more of the associated listed items. It will also be understood that, although the terms first, second, etc. may be used herein to describe various elements, these elements should not be limited by these terms, as these terms are only used to distinguish one element from another unless stated otherwise or the context indicates otherwise.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As defined herein, the term “partial amino acid sequence listing” means a listing of an amino acid sequence in which one or more amino acids in the amino acid sequence are unknown.

As defined herein, the term “complete amino acid sequence listing” means a listing of an amino acid sequence in which one or more amino acids, which are unknown in a partial amino acid sequence listing, are predicted.

As defined herein, the term “natural language” means a human spoken language that evolved naturally among humans. As the term “natural language” is defined herein, an artificial language is not a “natural language.” As the term “natural language” is defined herein, a computer programing language is not a “natural language.”

As defined herein, the term “linear projection” means generation of a second vector from a first vector by multiplying the first vector by a linear projection matrix.

As defined herein, the term “amino acid sequence vector” means a vector in which amino acids of an amino acid chain are represented by tokens.

As defined herein, the term “partial amino acid sequence vector” means an amino acid sequence vector in which one or more amino acids represented by the amino acid sequence vector are unknown or masked.

As defined herein, the term “predicted amino acid sequence vector” means an amino acid sequence vector in which one or more amino acids represented by the amino acid sequence vector are predicted.

As defined herein, the term “language vector” means a vector comprising a plurality of tokens for words of a natural language.

As defined herein, the term “responsive to” means responding or reacting readily to an action or event. Thus, if a second action is performed “responsive to” a first action, there is a causal relationship between an occurrence of the first action and an occurrence of the second action, and the term “responsive to” indicates such causal relationship.

As defined herein, the term “computer readable storage medium” means a storage medium that contains or stores program code for use by or in connection with an instruction execution system, apparatus, or device. As defined herein, a “computer readable storage medium” is not a transitory, propagating signal per se.

As defined herein, the term “data processing system” means one or more hardware systems configured to process data, each hardware system including at least one processor programmed to initiate executable operations and memory.

As defined herein, the term “processor” means at least one hardware circuit (e.g., an integrated circuit) configured to carry out instructions contained in program code. Examples of a processor include, but are not limited to, a central processing unit (CPU), an array processor, a vector processor, a digital signal processor (DSP), a field-programmable gate array (FPGA), a programmable logic array (PLA), an application specific integrated circuit (ASIC), programmable logic circuitry, and a controller.

As defined herein, the term “output” means storing in memory elements, writing to display or other peripheral output device, sending or transmitting to another system, exporting, or similar operations.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
generating, using a processor, a first language vector by performing a first linear projection on a partial amino acid sequence vector;
generating a second language vector by performing natural language processing on the first language vector;
generating a predicted amino acid sequence vector by performing a second linear projection on the second language vector; and
outputting a complete amino acid sequence listing based on the predicted amino acid sequence vector.

2. The method of claim 1, wherein the partial amino acid sequence vector represents a partial amino acid sequence listing.

3. The method of claim 1, wherein the performing the first linear projection on the partial amino acid sequence vector comprises multiplying the partial amino acid sequence vector by a first linear projection matrix.

4. The method of claim 3, further comprising:
performing machine learning by modifying coefficients of the first linear projection matrix.

5. The method of claim 3, wherein the performing the second linear projection on the second language vector comprises multiplying the second language vector by a second linear projection matrix.

6. The method of claim 5, further comprising:
performing machine learning by modifying coefficients of the second linear projection matrix.

7. The method of claim 5, further comprising:
performing machine learning by modifying coefficients of the first linear projection matrix and modifying coefficients of the second linear projection matrix.

8. A system, comprising:
a processor programmed to initiate executable operations comprising:
generating, using a processor, a first language vector by performing a first linear projection on a partial amino acid sequence vector;
generating a second language vector by performing natural language processing on the first language vector;
generating a predicted amino acid sequence vector by performing a second linear projection on the second language vector; and
outputting a complete amino acid sequence listing based on the predicted amino acid sequence vector.

9. The system of claim 8, wherein the partial amino acid sequence vector represents a partial amino acid sequence listing.

10. The system of claim 8, wherein the performing the first linear projection on the partial amino acid sequence vector comprises multiplying the partial amino acid sequence vector by a first linear projection matrix.

11. The system of claim 10, further comprising:
performing machine learning by modifying coefficients of the first linear projection matrix.

12. The system of claim 10, wherein the performing the second linear projection on the second language vector comprises multiplying the second language vector by a second linear projection matrix.

13. The system of claim 12, further comprising:
performing machine learning by modifying coefficients of the second linear projection matrix.

14. The system of claim 12, further comprising:
performing machine learning by modifying coefficients of the first linear projection matrix and modifying coefficients of the second linear projection matrix.

15. A computer program product, comprising:
one or more computer readable storage mediums having program code stored thereon, the program code stored on the one or more computer readable storage mediums collectively executable by a data processing system to initiate operations including:
generating a first language vector by performing a first linear projection on a partial amino acid sequence vector;
generating a second language vector by performing natural language processing on the first language vector;
generating a predicted amino acid sequence vector by performing a second linear projection on the second language vector; and
outputting a complete amino acid sequence listing based on the predicted amino acid sequence vector.

16. The computer program product of claim 15, wherein the partial amino acid sequence vector represents a partial amino acid sequence listing.

17. The computer program product of claim 15, wherein the performing the first linear projection on the partial amino acid sequence vector comprises multiplying the partial amino acid sequence vector by a first linear projection matrix.

18. The computer program product of claim 17, wherein the program code is executable by the data processing system to initiate operations further comprising:
performing machine learning by modifying coefficients of the first linear projection matrix.

19. The computer program product of claim 17, wherein the performing the second linear projection on the second language vector comprises multiplying the second language vector by a second linear projection matrix.

20. The computer program product of claim 19, wherein the program code is executable by the data processing system to initiate operations further comprising:
performing machine learning by modifying coefficients of the second linear projection matrix.
